(19) United States
(12) Patent Application Publication
(10) Pub. No.: US 2018/0369362 A1
(43) Pub. Date: Dec. 27, 2018

(54) INFECTIOUS BRONCHITIS VIRUS VACCINE USING NEWCASTLE DISEASE VIRAL VECTOR

(71) Applicant: UNIVERSITY OF MARYLAND, COLLEGE PARK, College Park, MD (US)

(72) Inventors: Shin-Hee Kim, Greenbelt, MD (US); Siba Samal, Hyattsville, MD (US)

(21) Appl. No.: 16/016,540

(22) Filed: Jun. 22, 2018

Related U.S. Application Data
(60) Provisional application No. 62/523,645, filed on Jun. 22, 2017.

(51) Int. Cl. A61K 39/145; C07K 14/005; C12N 9/24

(52) U.S. Cl. CPC A61K 39/145; C07K 14/005; C12N 9/2402; A61K 2039/5256; C12N 2760/18141; C12N 2760/16134; C12N 2760/18134

(57) ABSTRACT

Provided are compositions and methods that involve recombinant Newcastle disease viruses (rNDVs), and recombinant vectors that encode them. The rNDVs comprise and/or encode a combination of at least two proteins that are hemagglutinin (HA), neuraminidase (NA) protein, matrix 1 protein (M1), or nonstructural 1 protein (NS1). The HA, NA, protein, M1, and nonstructural 1 protein (NS1) are from Avian Influenza virus (AIV). Method are provided and involve administering an immunologically effective amount rNDV to avian animals to stimulate a protective immune response the rNDV administration constitutes a first (prime) immunization, which can be followed by a second (boost) administration with an avirulent NVD that may include/encode an AIV HA. The avian animals can survive challenges from pathogenic and highly pathogenic NVD and AIV.

### CROSS REFERENCE TO RELATED APPLICATIONS

This application claims priority to U.S. Provisional Application No. 62/523,645 filed on Jun. 22, 2017, the disclosure of which is hereby incorporated by reference.

### FIELD OF THE DISCLOSURE

The disclosure generally relates to vaccines. More particularly the disclosure generally relates to modified Newcastle disease viruses (NDVs) expressing proteins of avian influenza virus (AIV).

### BACKGROUND

Influenza viruses belong to the family Orthomyxoviridae with segmented, negative sense, single-strand RNA genomes [1]. Among the five genera, the Influenzavirus A genus contain eight gene segments and encode at least 10 proteins: polymerase basic 1 (PB1), PB2, polymerase acid (PA), hemagglutinin (HA), nucleoprotein (NP), neuraminidase (NA), matrix 1 (M1), M2, nonstructural 1 (NS1) and 2 (NS2). All the avian influenza viruses (AIVs) are classified into the genus Influenzavirus A. Among the 18 known HA subtypes of Influenza A viruses, H5 and H7 subtypes have caused high levels of morbidity and mortality in chickens and other terrestrial poultry. Vaccines have been used to control AIV infections in the fields [2]. Inactivated, oil adjuvanted, whole virus vaccines are the most common vaccines available for AIV. However, poor quality vaccines and inappropriate application have led to vaccine failures in the field. The use of attenuated live influenza vaccines in poultry is not recommended due to the potential risk of reassortment or mutations [3].

Newcastle disease virus (NDV) is also an economically important respiratory pathogen in poultry industry. NDV strains are classified into three pathontypes: lentogenic (avirulent), mesogenic (moderately virulent), and velogenic (virulent) [4]. Avirulent NDV strains, such as LaSota and B1, are used as live attenuated vaccines to control Newcastle disease in poultry worldwide [5]. Furthermore, they have been used as promising vaccine vectors for human and veterinary uses [6]. NDV can be an ideal vaccine vector for development of an AI vaccine, since NDV replicates efficiently and induces strong local and systemic immune responses at the respiratory tract [7]. However, pre-existing immunity to the vector due to vaccination against NDV has limited the protective efficacy of NDV vectored vaccines in the field [8]. Thus, there is an ongoing need for improved NDV vaccines. The present disclosure is pertinent to this need.

### SUMMARY OF THE DISCLOSURE

The present disclosure provides recombinant vectors, recombinant vaccines, recombinant viruses, and methods of using the same. In embodiments, the disclosure provides chimeric Newcastle disease virus (NDV) comprising Avian Influenza virus (AIV) proteins/antigens. In embodiments, the vaccine viruses comprise chimeric NDVs expressing a combination of AIV hemagglutinin (HA), neuraminidase (NA) protein, matrix 1 protein, and nonstructural 1 protein. Comparison of their protective efficacy between a single and prime-boost immunizations indicated that prime immunization of 1-day-old SPF chicks with the vaccine viruses followed by boosting with a conventional NDV vector (strain LaSota) expressing the HA protein provided complete protection of chickens against mortality, clinical signs and virus shedding. Further verification of this heterologous prime-boost immunization using commercial broiler chickens indicated that a sequential immunization of chickens with chimeric NDV vector expressing the HA and NA proteins following the boost with NDV vector expressing the HA protein demonstrated utility of the invention at least in terms of vaccination against highly pathogenic avian influenza virus (HPAIV) and against highly virulent NDVs. Thus, in embodiments the disclosure comprises sequential vaccinations, i.e., heterologous prime-boost immunizations, as described herein. All vectors, recombinant viral genomes, antigenomes, and methods of administering the vaccines described herein are encompassed by this disclosure. In embodiments, the compositions described herein are administered to an avian animal that is an embryo, a fledgling, or an adult avian animal. In embodiments, the avian animal is a chicken, such as Gallus gallus. In embodiments, populations or sub-populations of avian animals are vaccinated to promote, for example, herd immunity. In embodiments, the disclosure provides for more a more efficient/effective immunization relative previously available compositions and methods, at least insofar as reducing or eliminating maternal antibody interference. Furthermore, and without intending to be constrained by any particular theory, it is considered that the present vaccine preparations are suitable for alternative routes of the administration, such as through drinking water, which provides for efficient administration of the vaccines relative to previous versions that employ in ovo or subcutaneous methods.

In embodiments, the disclosure thus comprises a recombinant Newcastle disease virus (rNDV) comprising and/or encoding a combination of at least two proteins that are hemagglutinin (HA), neuraminidase (NA) protein, matrix 1 protein (M1), or nonstructural 1 protein (NS1). In embodiments, the HA, NA, protein, M1, and nonstructural 1 protein (NS1) are from Avian Influenza virus (AIV). In embodiments, the rNDV comprises the AIV and HA and NA. In an embodiment, the only proteins from the AIV comprise the HA and the NA. In embodiments, the NA, M1 or NS1 is between M and F genes in the chimeric NDV, and wherein the rNDV further comprises the HA.

In an embodiment, the disclosure comprises administering an immunologically effective amount rNDV to avian animals to stimulate a protective immune response against at least one of pathogenic NDV or pathogenic Avian Influenza virus (AIV), wherein the rNDV comprises and/or encodes a combination of at least two proteins that are AIV HA, NA, M1, or NS1. In embodiments, the rNDV comprises the AIV HA and NA. In embodiments, the administration of the rNDV administration comprises a first (prime) immunization. In embodiments, the disclosure comprises a second (boost) administration with an avirulent NVD, wherein the avirulent NVD may further comprise and/or encode AIV HA. In an embodiment, the avirulent NVD comprises a LaSota strain NVD. In embodiments, the prime boost approach results in at least one of the following effects; the avian animals survive exposure to pathogenic AIV or pathogenic NDV, or both, or exhibit reduced clinical signs of AIV or NDV infection, or both, or exhibit reduced AIV, reduced NDV shedding, or both. In embodiments, the avian animals survive exposure to highly pathogenic AIV, or highly virulent NDV, or both. Exposure means the animals were exposed to sufficient pathogenic AIV or NDV such that, in the absence of the vaccine, the animals would be infected, or at least sickened, and/or exhibited signs of infection, and/or killed by the AIV or the NDV. In embodiments, avian animals vaccinated according to this disclosure have high levels of maternal antibody that reduce effectiveness of previously available vaccines.

### BRIEF DESCRIPTION OF THE FIGURES

For a fuller understanding of the nature and objects of the disclosure, reference should be made to the following detailed description taken in conjunction with the accompanying figures.

FIG. 1. Generation of chimeric NDV vectored vaccine viruses expressing the H5N1 HPAIV HA gene in combination with NA, M1, or NS1. (A) A full-length antigenomic cDNA of chimeric NDV/HA was modified by inserting each gene flanked by the NDV gene-start and gene-end signals into the intergenic region between the M and F genes. Ectodomains of the F and HN genes derived from APMV-2 are shown as white rectangle. (B) To evaluate the syncytium formation of chimeric vaccine viruses, DF1 cells in six-well plates were infected with the indicated viruses at a multiplicity of infection (MOI) of 0.01 PFU/cell, incubated for 72 h, and conducted immunoperoxidase staining using antiserum against the N protein of NDV, with viral antigen stained red. (C) The growth kinetics was determined by infecting DF1 cells with each virus at an MOI of 0.01. (D) Expression of H5N1 HPAIV HA, NA, M1, and NS1 proteins by NDV vectors in DF1 cells were analyzed by Western blot. DF1 cells were infected with each virus at MOI 1, and cell lysates were collected at 24 h post-infection for Western blot analysis.

FIG. 2. Immunogenicity of chimeric NDV vectored vaccines in SPF chickens. SPF chickens were immunized with each vaccine virus (10⁶ pfu/ml) by the oculonasal route. In chickens immunized with a single dose (A and C), serum samples were collected at 3 wpi prior to challenge. For prime-boost immunization group (B and C), the immunized SPF chickens with each vaccine virus were boosted oculonasally with LaSota/HA at 2 weeks post immunization. The serum samples were collected at 2 weeks post-boost. Virus-specific antibodies were determined by a hemagglutination inhibition (HI) assay using LaSota, chimeric NDV, and VN/04 H5N1.

FIG. 3. Protective efficacy of NDV vectored vaccines in SPF chickens. The prime-boost immunization groups of SPF chickens were challenged with 10⁶ ELD₅₀ of HPAIV VN/04 H5N1 by the oculonasal route. The mortality (A) and virus shedding (B) in challenged SPF chickens were evaluated. Oral and cloacal swabs were collected from the chickens at 3 days post challenge, and shedding of the challenge virus was determined by inoculating clarified swab samples into 9-day-old SPF embryonated chicken eggs and conducting HA assay.

FIG. 4. Immunogenicity of chimeric NDV vectored vaccines in broiler chickens. One-day-old broiler chicks were immunized with each vaccine virus (10⁶ pfu/ml) by the oculonasal route and boosted with LaSota/HA oculonasally at 2 wpi. In addition, one group of 2-week-old broiler chickens were oculonasally immunized with a single dose of LaSota/HA. Serum samples were collected prior to boosting (A) and challenge (B). Virus-specific antibodies were determined by a hemagglutination inhibition assay using LaSota (A and B), chimeric NDV (A and B), and VN/04 H5N1 (C).

FIG. 5. Protective efficacy of NDV vectored vaccines in broiler chickens. The prime-boost immunization groups of broiler chickens were challenged oculonasally with 10⁶ ELD₅₀ of HPAIV VN/04 H5N1 (A) and highly virulent NDV strain GB Texas (200 μlof 100 CLD₅₀) (B). The mortality and shedding of challenge virus in broiler chickens were evaluated. Oral and cloacal swabs were collected from the chickens at 3 days post challenge, and shedding of the challenge virus was determined by inoculating clarified swab samples into 9-day-old SPF embryonated chicken eggs and conducting HA assay.

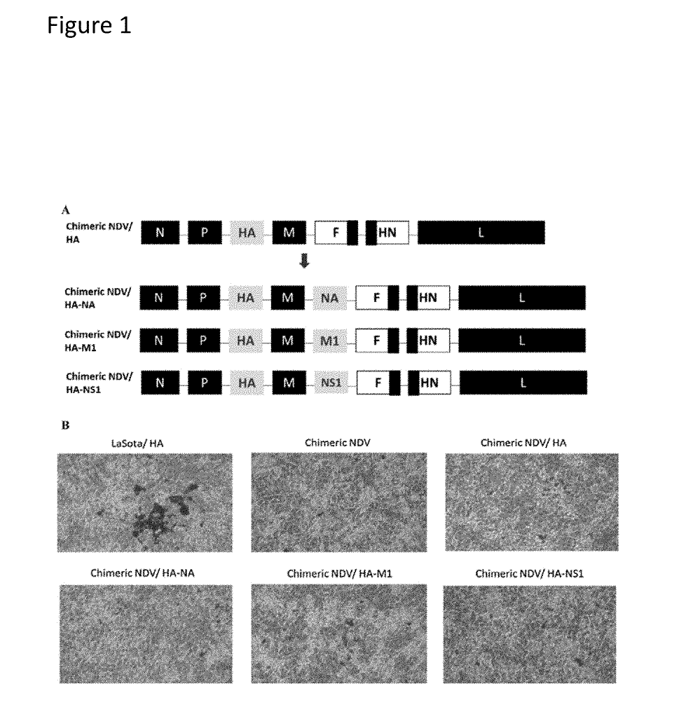

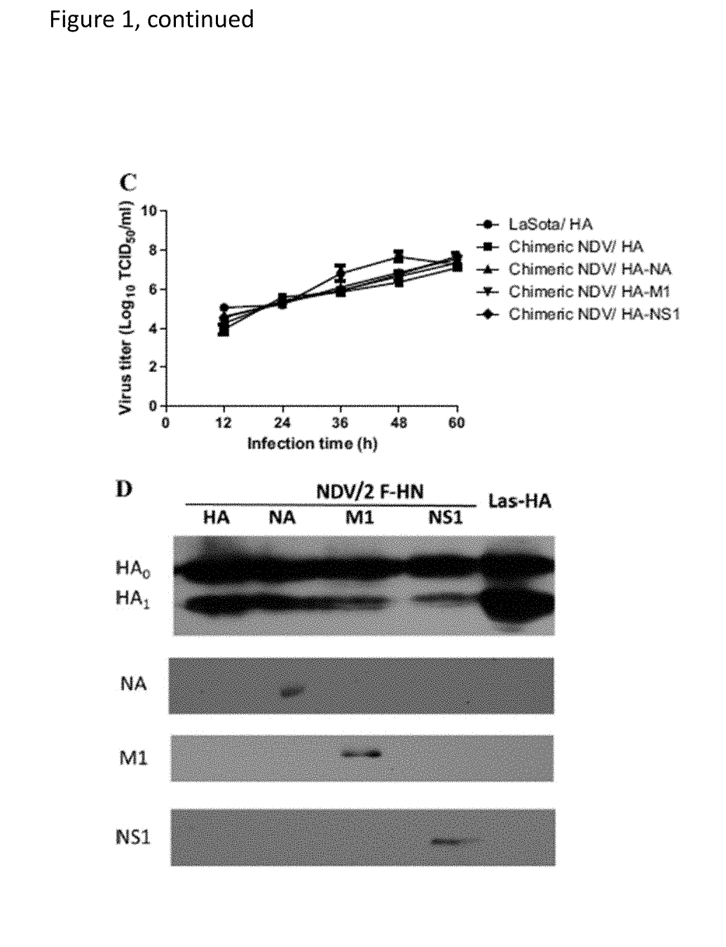

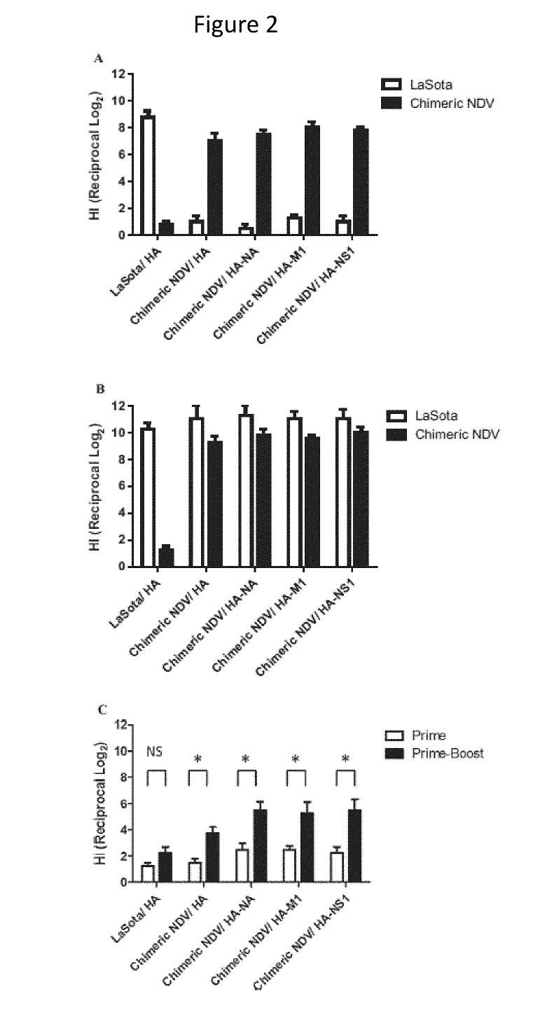

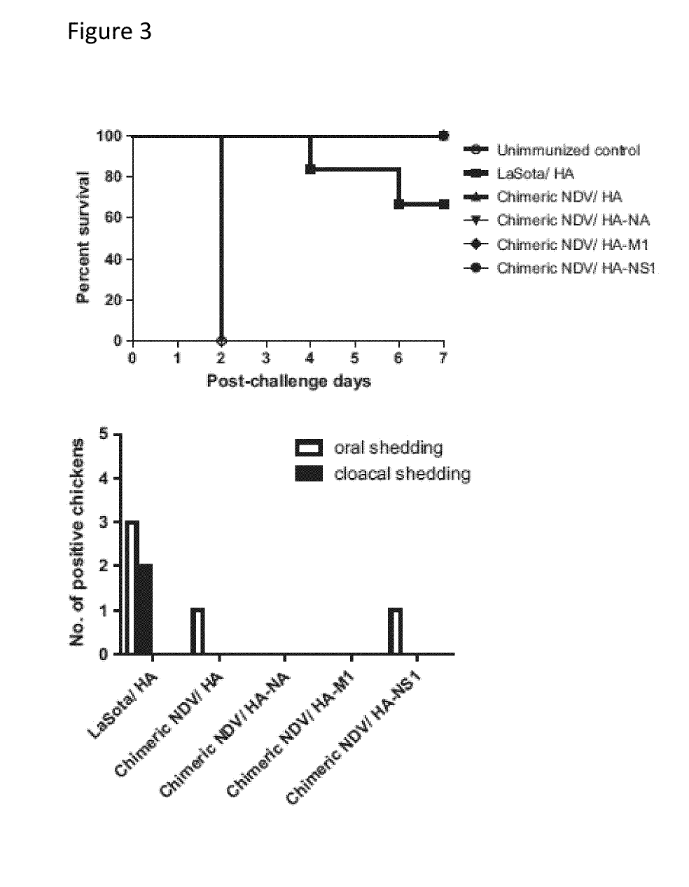

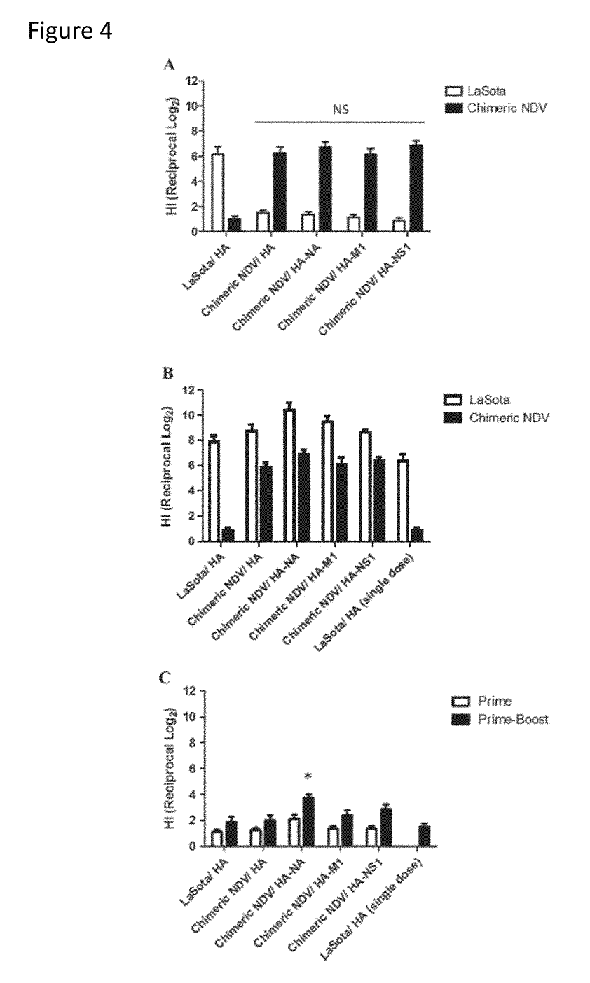

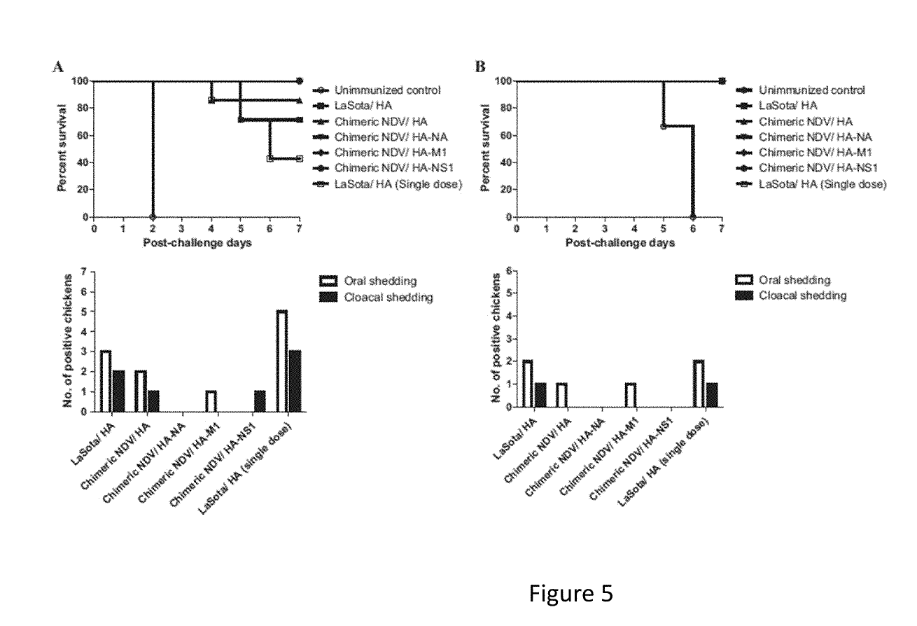

### DETAILED DESCRIPTION OF THE DISCLOSURE

Unless defined otherwise herein, all technical and scientific terms used in this disclosure have the same meaning as commonly understood by one of ordinary skill in the art to which this disclosure pertains.

Every numerical range given throughout this specification includes its upper and lower values, as well as every narrower numerical range that falls within it, as if such narrower numerical ranges were all expressly written herein.

Ranges of values are disclosed herein. Unless otherwise stated, the ranges include all values to the magnitude of the smallest value (either lower limit value or upper limit value) and ranges between the values of the stated range.

The present disclosure relates to modified viral vectors to control infection by at least one of AIV, HPAIV, or highly virulent NDVs.

The disclosure includes all polynucleotide and amino acid sequences described herein, and every polynucleotide sequence referred to herein includes its complementary DNA sequence, and also includes the RNA equivalents thereof to the extent an RNA sequence is not given. Every DNA and RNA sequence disclosed herein is encompassed by this disclosure, including but not limited to sequences encoding all viral and recombinant proteins that are described here. Where a DNA sequence is provided or would otherwise be known to those skilled in the art it may be a cDNA sequence of a viral negative sense genome; the skilled artisan can from the cDNA sequence readily envision the negative sense strand in its RNA form. The disclosure includes all negative strand viral genome sequences, and all complementary (cRNA) sequences thereof. In embodiments, the RNA sequences can comprise non-templated G residues arising from RNA editing. In embodiments, the disclosure includes recombinant polynuculeotides comprise an engineered paramyxovirus antigenome. In embodiments, more than one copy of any protein/antigen described herein can be included in the vaccine formulations of this disclosure. In embodiments, a protein expressed from a construct described herein may comprise an uncleaved protein precursor, which may be subsequently cleaved. Any combination of proteins produced by the chimeric vaccine constructs of this disclosure can be from NDV, or AIV, or the proteins can be from NDV and AIV. In embodiments, an indication that an chimeric NDV encodes a protein means that the protein is present in the NDV, and/or expressed in cells infected by the NDV. In embodiments, chimeric NDVs described herein comprise recombinant NDVs (rNDVs).

NDV belongs to the genus Avulavirus in the family Paramyxoviridae. Paramyxovirus pathogens include measles virus, mumps virus, human respiratory syncytial virus, and the zoonotic paramyxoviruses Nipah virus and Hendra virus. The genus Avulavirus includes at least 13 serotypes of Avian Avulaviruses (AAvV). All strains of NDV belong to Avulavirus—type 1(AAvV-1), but the other serotypes can also be used as modified or unmodified vaccine vectors. The disclosure includes use of other such modified or unmodified avulaviruses to achieve multivalent immunizations that include an immune response to any of AIV, HPAIV, or highly virulent NDVs.

With respect to NDV, virulent NDV strains cause a fatal neurological disease in chickens. NDV strain LaSota has been used as a safe and effective live vaccine for more than 60 years. Recombinant NDV (rNDV) strain LaSota has been evaluated as a vaccine vector against several avian pathogens.

The Examples that follow demonstrate generation of recombinant rNDVs. In this regard, conventional NDV vectored vaccines have shown to be effective in protecting SPF chickens against HPAIV [7, 12-14]. In the field, recombinant NDVs are approved as bivalent vaccines in poultry against NDV and AIV, and are used in China for H5N1 HPAIV (Chen 2009 Ge 2007) and in Mexico for the H5N2 LPAIV (Villarreal 2009). However, almost all commercial chickens and turkeys are vaccinated against NDV, and chicks typically have high levels of maternal antibody, which could interfere with the protective efficacy of the vaccine in the field (Spackman and Pantin-Jackwood, 2014). This obstacle was also observed with vaccination with other vector viruses, such as fowlpox virus (Swayne 2000) and turkey herpesvirus (Kilany 2015; Rauw 2012), creating limitation on their mass vaccination and replacement of inactivated vaccines.

Accordingly, an aspect of this disclosure comprises NDV vectored AI vaccine strategies that provide early protection to short-lived broiler chickens by overcoming pre-existing antibody against NDV. In this disclosure, due to serological distance of chimeric NDV from NDV, we could reduce shedding of challenge virus in SPF chickens by heterologous prime-boost immunization. Compared to inactivated AI vaccines, the vaccination strategies described herein provides improved immunization with a convenient platform. First, use of serologically different live vectored vaccines can be cost-effective and applicable for mass vaccination through spray or drinking water. Second, the presently provides approaches can be advantageous with good immune response to a replicating virus. Therefore, vaccination strategies using the modified/chimeric viruses described herein is practical and effective for use in the field.

The protective efficacy of experimental vaccines has been mostly evaluated by immunizing SPF chickens. These experimental studies cannot completely simulate field conditions [16-17]. Consequently, there has been discrepancy in protective efficacy of vaccine trials between the laboratory and field conditions. Therefore, we further verified the protective efficacy of the present vaccination strategy in broiler chickens. As demonstrated in the Examples and figures of this disclosure, one-day-old chicks were retrieved from a parent flock in a commercial hatchery that had received routine vaccination, including ND vaccine. We found discrepancy in our vaccine trials between SPF and broiler chickens. In a previous study, single immunization of 2-week-old SPF chickens with LaSota/HA showed efficient protection against HPAIV in SPF chickens [13, 14], but this immunization provided insufficient protection against HPAIV in broiler chickens. Increase to two doses of immunization still did not provide complete protection of chickens against mortality and virus shedding in broiler chickens. Therefore, the present disclosure indicates that a sequential administration of LaSota vector does not significantly enhance the levels of protective immunity against HPAIV in broiler chickens. However, the heterologous prime-boost vaccination strategy descried herein efficiently provides complete protection against HPAIV.

Protective efficacy study in broiler chicken also enabled identification of what is considered, without intending to be constrained by any particular theory, the best vaccine candidate (chimeric NDV/HA-NA) against HPAIV. During a natural AIV infection, antibodies against both the surface proteins of HA and NA are generated. Antibodies against HA inhibit the attachment to the sialic acid-containing host cell receptor and inhibit fusion between viral and host cell membranes [18]. Antibodies to the NA protein can impede its receptor-destroying function, thus reducing virus replication by inhibiting virus release from infected cells. Therefore, the HA and NA proteins of target influenza strains have been expressed simultaneously in many vaccine development studies [19, 20]. Previously, vectored and subunit vaccines have shown that the HA protein provides better protective immunity than the NA protein [21]. To combat this, modified/chimeric viral vaccines described herein simultaneously express the HA protein as a major protective antigen and the NA protein as a minor antigen by taking an advantage of the nature of polar gradient of NDV transcription [22]. It is expected that the vaccination strategies of this disclosure will be highly beneficial to the poultry industry. The HA and NA genes in chimeric vector and the HA gene in NDV vector can be easily replaced with those of currently circulating HPAIV strains. Results presented herein also show that boosting 2-week-old chickens with LaSota/H5 can also serve as a dual vaccination approach against HPAIV and highly virulent NDV. In hatchery, Newcastle disease vaccination is routinely conducted for commercial broiler chickens using live attenuated NDV strains B1 or LaSota at 1 day or 14-21 days of hatching. Therefore, the presently provided vaccination approach can be feasible for use as a routine vaccination program for commercial broiler chickens. Furthermore, this vaccination strategy can be used for other avian pathogens, such as infectious laryngotracheitis and infectious bronchitis viruses. Since the presently provided chimeric NDV vaccine is highly attenuated and is safer than conventional NDV vector, this also makes it suitable for in ovo vaccination, a mass vaccination approach in poultry industry. Therefore, it is expected that the chimeric NDV constructs provided herein can be suitable AI vaccine vectors for many applications with a practical strategy for rapid, efficient, and economical immunization of chickens in the field.

It will be apparent from the foregoing that various embodiments of this disclosure provide chimer NVDs that include AIV antigens. The amino acid and nucleotide sequences of a variety of strains of NVD and AIV viruses are known in the art, and it is contemplated that the segments of such proteins as described herein can be used and/or modified for use with embodiments of this disclosure. In certain embodiments, a protein or segment thereof of this disclosure may differ from a reference sequence. Thus, in certain examples the disclosure comprises a modified segment of a viral protein that comprises at least one amino acid change relative to the unmodified (wild type) counterpart. In certain examples more than one amino acid change can be included. Such changes can comprise conservative or non-conservative amino acid substitutions, insertions, and deletions, provided the modified sequence retains or improves on the capability to be used to stimulate an immune response.

To produce the viral particles, the viral particles themselves, or DNA/cDNA or RNA or cRNA encoding the required set of proteins can be introduced directly into producer cells, and shed viral particles can be isolated from the cells. In embodiments, one or more expression vectors can be used to produce the viral particles. In this regard, a variety of suitable expression vectors known in the art can be adapted to produce the modified paramyxovirus particles of this disclosure. In general, the expression vector comprises sequences that are operatively linked with the sequences encoding the viral particle proteins that comprise NDV and AIV genes, or another suitable insertion site. A particular polynucleotide sequences is operatively-linked when it is placed in a functional relationship with another polynucleotide sequence. For instance, a promoter is operatively-linked to a coding sequence if the promoter affects transcription or expression of the coding sequence. Generally, operatively-linked means that the linked sequences are contiguous and, where necessary to join two protein coding regions, both contiguous and in reading frame. However, it is well known that certain genetic elements, such as enhancers, may be operatively-linked even at a distance, i.e., even if not contiguous, and may even be provided in trans. Promoters present in expression vectors that are used in the present disclosure may be endogenous or heterologous to the host cells, and may be constitutive or inducible. Expression vectors can also include other elements that are known to those skilled in the art for propagation, such as transcription and translational initiation regulatory sequences operatively-linked to the polypeptide encoding segment. Suitable expression vectors may include, for example, an origin of replication or autonomously replicating sequence (ARS) and expression control sequences, an enhancer and other regulatory and/or functional elements, such as ribosome-binding sites, RNA splice sites, polyadenylation sites, transcriptional terminator sequences, and mRNA stabilizing sequences, as well as a wide variety of selectable markers.

In certain embodiments, viral particles can be produced using a set of plasmids, which may be used in conjunction with a cDNA, such as an antigenome of an NDV that is modified to include a polynucleotide sequence that encodes (or can be transcribed to encode) an AIV protein, the coding sequence for which can be placed between the NDV M and F genes in, for example, a chimeric NDV containing the HA gene. In embodiments, a full length cDNA of a chimeric NDV can be co-transfected into suitable cells with one or more plasmids that express, for example, the N, P and L genes of NDV so that recombinant NDV particles can be produced by the cells, and recovered. In this regard, the expression vectors can be introduced into the host producer cells by any method known in the art. These methods vary depending upon the type of cellular host, and include but are not limited to transfection employing cationic liposomes, electroporation, calcium chloride, rubidium chloride, calcium phosphate, DEAE-dextran, or other substances as will be apparent to the skilled artisan. In embodiments the viral particles are produced using chicken embryo fibroblast cells, such as DF-1 cells, or mammalian cells, such as HEp-2 cells. In embodiments, the rNDVs are produced in embryonated, specific-pathogen-free (SPF) eggs. Methods of making the modified virus particles are accordingly included, and generally comprise introducing polynucleotides encoding the viral genome and viral proteins virus into suitable producer cells, and recovering shed virus from them. Thus, cells and cell cultures that harbor polynucleotides encoding the modified paramyxoviruses of this disclosure are included, as are isolated and/or purified modified paramyxovirus viral particle preparations. The particles can be purified to any desired degree of purity using standard approaches, such as density gradient separation or commercially available kits used to purify enveloped viruses or exosomes.

In certain aspects the disclosure includes a pharmaceutical formulation comprising modified paramyxovirus particles as described herein. The form of pharmaceutical preparation is not particularly limited, but generally comprises modified viral particles and at least one inactive ingredient. In certain embodiments suitable pharmaceutical compositions can be prepared by mixing any one type of the particles, or combination of distinct types of particles, with a pharmaceutically-acceptable carrier, diluent or excipient, or immune response regulator, or an antibiotic, and suitable such components are well known in the art. Some examples of such carriers, diluents and excipients can be found in: Remington: The Science and Practice of Pharmacy (2005) 21st Edition, Philadelphia, Pa. Lippincott Williams & Wilkins, the disclosure of which is incorporated herein by reference. In embodiments, a vaccine formulation is provided. In embodiments, a formulation of this disclosure is provided as an effervescent tablet, a pellet, or in a lyophilized form. The pharmaceutical composition can be also include, for example, any suitable adjuvant.

In embodiments, the rNDV particles are administered to avian animals using any suitable route. In embodiments, the particles are provided as vaccines and can be administered orally, intranasally, intraocularly or parenterally, e.g. by intramuscular or subcutaneous injection. In embodiments, the vaccine formulations are administered via an oculanasal route, thus conjunctival and intranasal routes are included. In embodiments, the vaccine is administered in drinking water, or as an aerosol or a spray. In embodiments, a vaccine formulation of this disclosure is administered in ovo, as an eye drop, or injection, such as a subcutaneous or wing-web injection. In embodiments, the vaccines are administered through drinking water, or by spraying. In embodiments, the disclosure includes an article of manufacture, such as a kit, the kit comprising rNTDs as described herein in any suitable form, wherein the rNTDs are comprised within one or more containers, and wherein the article or kit comprises printed material, such as a label or insert that includes instructions for using the rNTDs for vaccination of avian animals.

In embodiments, immunologically effective amount rNDV particles are administered. Immunologically effective as used herein means an amount that results in production of neutralizing antibodies against any of the pathogenic viruses discussed herein, and/or results in reduced shedding of the challenge virus, and or can provide full or partial protection against mortality, or can reduce clinical signs of infection.

In embodiments, an immunologically effective amount results in preventing and/or lessening of clinical disease and/or mortality when challenged with a virulent virus. Immunological protection can be durable, and last for days, weeks or months, or longer, after vaccination, and such vaccinations can be effective to elicit such protection after a single dose, or multiple doses. Booster vaccinations can be used according to this disclosure and include the heterologous Prime-Boost strategies described herein.

In embodiments, neutralizing antibodies are produced. The term “neutralizing antibody” refers to an antibody or a plurality of antibodies that inhibits, reduces or completely prevents viral infection. Whether neutralizing antibodies are produced can be determined by in vitro assays that are known in the art.

In embodiments, viral load in the vaccinated animals is reduced. Viral load can be determined according to methods known to those skilled in the art.

In embodiments, an embryo infective dose (EID) is used.

In embodiments, an rNDV is modified such that it is less pathogenic than an un-modified NDV, and thus may comprise or be derived from an avirulent rNDV, or it may be provided as an attenuated or inactivated vaccine. In embodiments, an rNDV for use in vaccines of this disclosure are derived from NDV strains from the class II genotype I. In embodiments, an rNDV that is resistant to heat is used.

In embodiments, the avian animals to which compositions of this disclosure are administered are any type of poultry. In embodiments, the avian animals are Galliformes and thus include any members of the order of heavy-bodied ground-feeding birds that includes turkey, grouse, chicken, New World quail and Old World quail, ptarmigan, partridge, pheasant, junglefowl and the Cracidae. In embodiments, the avian animals are domesticated fowl, including but not limited to domesticated chickens and turkeys. In embodiments, the chickens are Gallus gallus, such as Gallus gallus domesticus. In embodiments, the chickens are roosters or hens. In embodiments, the avian animals are adults, juveniles, or embryos. In an embodiment, a composition of this disclosure is applied to eggs. In embodiments, vaccines of this disclosure administered to a population of avian animals, i.e., a flock. In embodiments, from 50-85% or more members of the flock are vaccinated to achieve, for example, herd or flock immunity.

The results showed that

The Examples are presented to illustrate the present disclosure. They are not intended to limiting in any matter.

### EXAMPLE 1

TABLE 1
Pathogenicity of parental and chimeric NDV vectored vaccine
viruses in embryonated eggs and in chicks

| Virus | MDT^{a} | ICPI^{b} |
|---|---|---|
| LaSota | 115 h | 0.00 |
| Chimeric NDV | >198 h | 0.00 |
| LaSota/HA | 123 h | 0.00 |
| Chimeric NDV/HA | >198 h | 0.00 |
| Chimeric NDV/HA-NA | >198 h | 0.00 |
| Chimeric NDV/HA-M1 | >198 h | 0.00 |
| Chimeric NDV/HA-NS1 | >198 h | 0.00 |

^{a}Mean embryo death time (MDT): the mean time (h) for the minimum lethal dose of virus to kill all of the inoculated embryos. Pathotype definition: virulent strains, <60 h; intermediate virulent strains, 60 to 90 h; avirulent strains, >90 h.
^{b}Pathogenicty of NDV in 1-day-old SPF chicks was evaluated by the ICPI assay. Pathotype definition: virulent strains, 1.5-2.0; intermediate virulent strains, 0.7-1.5; and avirulent strains, 0.0-0.7.

References: The following reference listing is not intended to be an indication that any of the references are material to patentability.
[1] Palese P, Shaw M L. Orthomyxoviridae: the viruses and their replication. D. M. Knipe, P. M. Howley (Eds.), Fields Virology (5th ed.), Lippincott Williams & Wilkins. 2007; p. 1647-1689.
[2] Swayne D E, Pavade G, Hamilton K, Vallat B, Miyagishima K. Assessment of national strategies for control of high-pathogenicity avian influenza and low-pathogenicity notifiable avian influenza in poultry, with emphasis on vaccines and vaccination. Rev Sci Tech 2011; 30: 839-870.
[3] Fuchs W, Römer-Oberdörfer A, Veits J, Mettenleiter T C. Novel avian influenza virus vaccines. Revue Scientifique et Technique, Office International des Epizooties 2009; 28: 319-332.

[4] Alexander D J. Newcastle disease,. In: Purchase H G, Arp L H, Domermuth C H, Pearson J E. A laboratory manual for the isolation and identification of avian pathogens, 3rd ed. The American Association of Avian Pathologists, Kendall/Hunt Publishing Company, Dubuque, Iowa. 1989; p.114-120.
[5] Samal S K. Newcastle disease and related avian paramyxoviruses, In: Samal, S. K., editor. The biology of paramyxoviruses. Caister Academic Press. 2011; p. 69-114.
[6] Kim S H, Samal S K. Newcastle disease virus as a vaccine vector for development of human and veterinary vaccines. Viruses 2016; 8: 183.
[7] DiNapoli J M, Yang L, Suguitan A, Elankumaran S, Dorward D W, Murphy B R, Samal S K, Collins P L, Bukreyev A. Immunization of primates with a Newcastle disease virus-vectored vaccine via the respiratory tract induces a high titer of serum neutralizing antibodies against highly pathogenic avian influenza virus. J Virol 2007; 81: 11560-11568.
[8] Spackman E, Pantin-Jackwood M J. Practical aspects of vaccination of poultry against avian influenza virus. Veterinary Journal 2014; 202: 408-415.
[9] Kim S H, Paldurai A, Samal S K. A novel chimeric Newcastle disease virus vectored vaccine against highly pathogenic avian influenza virus. Virology 2017; 503: 31-36.
[10] Huang Z, Krisnamurthy S, Panda A, Samal S K. High-level expression of a foreign gene from the 3′ proximal first locus of a recombinant Newcastle disease virus. J Gen Virol 2001; 82: 1729-1736.
[11] Reed L J, Muench H A. A simple method of estimating fifty percent endpoints. Am J Hyg 1938; 27: 493-497.
[12] Park M S, Steel J, Garcia-Sastre A, Swayne D, Palese P. Engineered viral vaccine constructs with dual specificity: avian influenza and Newcastle disease. Proc Natl Acad Sci USA 2006; 103: 8203-8208.
[13] Nayak B, Rout S N, Kumar S, Khalil M S, Fouda M M, Collins P L, Samal S K. Immunization of chickens with Newcastle disease virus expressing H5 hemagglutinin protects against highly pathogenic H5N1 avian influenza viruses. PLoS ONE 2009; 4: e6509.
[14] Kim S H, Paldurai A, Xiao S, Collins P L, Samal S K. Modified Newcastle disease virus vectors expressing the H5 hemagglutinin induce enhanced protection against highly pathogenic H5N1 avian influenza virus in chickens. Vaccine 2014; 32: 4428-4435.
[15] Maas R, Rosema S, van Zoelen D, Venema S. Maternal immunity against avian influenza H5N1 in chickens: limited protection and interference with vaccine efficacy. Avian Pathology 2011; 40: 87-92.
[16] Spackman E, Swayne D E. Vaccination of gallinaceous poultry for H5N1 highly pathogenic avian influenza: current questions and new technology. Virus Res 2013; 178: 121-132.
[17] Swayne D E, Pavade G, Hamilton K, Vallat B, Miyagishima K. Assessment of national strategies for control of high-pathogenicity avian influenza and low-pathogenicity notifiable avian influenza in poultry, with emphasis on vaccines and vaccination. Rev Sci Tech 2011; 30: 839-870.
[18] DiNapoli J M, Nayak B, Yang L, Finneyfrock B W, Cook A. et al. Newcastle disease virus-vectored vaccines expressing the hemagglutinin or neuraminidase protein of H5N1 highly pathogenic avian influenza virus protect against virus challenge in monkeys. J Virol 2010; 84: 1489-1503.
[19] Qiao C L, Yu K Z, Jiang Y P, Jia Y Q, Tian G B, Liu M, Deng G H, Wang X R, Meng Q W, Tang X Y. Protection of chickens against highly lethal H5N1 and H7N1 avian influenza viruses with a recombinant fowlpox virus co-expressing H5 haemagglutinin and N1 neuraminidase genes. Avian Pathol 2003; 32: 25-32
[20] Tian G, Zhang S, Li Y, Bu Z, Liu P, Zhou J, Li C, Shi J, Yu K, Chen H. Protective efficacy in chickens, geese and ducks of an H5N1-inactivated vaccine developed by reverse genetics. Virology 2005; 341: 153-162.
[21] Nayak B, Kumar S, DiNapoli J M, Paldurai A, Perez D R, Collins P L, Samal S K. Contributions of the avian influenza virus HA, NA, and M2 surface proteins to the induction of neutralizing antibodies and protective immunity. J Virol 2010; 84: 2408-2420.
[22] Bukreyev A, Skiadopoulos M H, Murphy B R, Collins P L. Nonsegmented negative-strand viruses as vaccine vectors. J Virol 2006, 80: 10293-10306.

Although the present disclosure has been described with respect to one or more particular embodiments and/or examples, it will be understood that other embodiments and/or examples of the present disclosure may be made without departing from the scope of the present disclosure.

1. A recombinant Newcastle disease virus (rNDV) comprising and/or encoding a combination of at least two proteins that are hemagglutinin (HA), neuraminidase (NA) protein, matrix 1 protein (M1), or nonstructural 1 protein (NS1).

2. The rNVD of claim 1, wherein the HA, NA, protein, M1, and nonstructural 1 protein (NS1) are from Avian Influenza virus (AIV).

3. The rNDV of claim 2, comprising the AIV and HA and NA.

4. The rNDV of claim 3, wherein the only proteins from the AIV comprise the HA and the NA.

5. The rNDV of claim 2, wherein NA, M1 or NS1 is between M and F genes in the chimeric NDV, and wherein the rNDV further comprises the HA.

6. A method comprising administering an immunologically effective amount recombinant Newcastle Disease Virus (rNDV) to avian animals to stimulate a protective immune response against at least one of NDV or Avian Influenza virus (AIV), wherein the rNDV comprises and/or encodes a combination of at least two proteins that are AIV hemagglutinin (HA), neuraminidase (NA) protein, matrix 1 protein (M1), or nonstructural 1 protein (NS1).

7. The method of claim 6, wherein the rNDV comprises the AIV and HA and NA.

8. The method of claim 6, wherein the only proteins from the AIV in the rNDV comprise the HA and the NA.

9. The method of claim 6, wherein the rNDV administration comprises a first (prime) immunization.

10. The method of claim 9, further comprising a second (boost) administration with an avirulent NVD.

11. The method of claim 10, wherein the avirulent NVD further comprises and/or encodes AIV HA.

12. The method of claim 11, wherein the avirulent NVD comprises a LaSota strain NVD.

13. The method of claim 10, wherein at least one of the following is true: the avian animals survive exposure to pathogenic AIV or pathogenic NDV, or both, or exhibit reduced clinical signs of AIV or NDV infection, or both, or exhibit reduced AIV, reduced NDV shedding, or both.

13. The method of claim 10, wherein the avian animals survive challenge with the pathogenic AIV.

14. The method of claim 13, wherein the AIV comprises a highly pathogenic AIV.

15. The method of claim 10, wherein the avian animals survive challenge with the pathogenic NVD.

16. The method of claim 15, wherein the NVD comprises highly virulent NDV.
